(19) United States
(12) Patent Application Publication
(10) Pub. No.: US 2021/0214665 A1
(43) Pub. Date: Jul. 15, 2021

(54) VESSEL FOR CULTURING CELLS

(71) Applicant: Thrive Bioscience, Inc., Wakefield, MA (US)

(72) Inventor: Alan Blanchard, Topsfield, MA (US)

(73) Assignee: Thrive Bioscience, Inc., Wakefield, MA (US)

(21) Appl. No.: 16/093,129

(22) PCT Filed: Apr. 12, 2017
(86) PCT No.: PCT/US2017/027239

§ 371 Date: Oct. 11, 2018

Related U.S. Application Data
(60) Provisional application No. 62/321,691, filed on Apr. 12, 2016.

(51) Int. Cl. C12M 1/00; C12M 1/06; C12N 5/078

(52) U.S. Cl. CPC C12M 23/26; C12N 5/0634; C12M 27/02

(57) ABSTRACT

Aspects of the invention relate to cell culture vessels and method for using the vessels to agitate and maintain cells in suspension. In some embodiments, the vessel has a body configured to hold a volume of liquid containing cells to be cultured, the vessel body having a deformable portion arranged to induce movement of the liquid sufficient to maintain the cells in suspension. In some embodiments, the deformable portion is in at least one of a base and sidewall of the vessel body. In some embodiments, the deformable portion is deformed according to a deformable pattern. In some embodiments, the deformable portion is deformed via a deformation plate having one or more moveable members that contact the deformable portion.

### CROSS REFERENCE TO RELATED APPLICATIONS

This application claims the benefit under 35 U.S.C. § 119(e) to U.S. Provisional Application Ser. No. 62/321,691, entitled “VESSEL FOR CULTURING CELLS,” filed on Apr. 12, 2016, which is herein incorporated by reference in its entirety.

### FIELD

Aspects relate to vessels for culturing cells and method for using such vessels to agitate and maintain the cells in suspension. Some aspects relate to using such a vessel in cell culture incubators and to methods for using such incubators.

### BACKGROUND

Cell culture vessels (e.g., spinner flasks and magnetic stirrers) are used to stir a liquid containing cells, the cells being agitated and maintained in suspension via rotation of an agitator. Cell culture is a useful technique in both research and clinical contexts. For example, culture of mammalian cells is often performed in order to establish clonal cell lines, tissue preparations, in vitro fertilization preparations, or to expand populations of stem cells.

### SUMMARY

Presently available vessels used to culture cells, such as spinner flasks and magnetic stirrers, use an agitator (e.g., an impeller or magnetic stir bar, respectively) to induce movement of the liquid, which, in turn, moves the cells within the liquid. This rotation may agitate the cells and also maintain the cells in suspension. In some embodiments, rotation of the agitator (and fluid) applies a shear stress on the cells. Unfortunately, cells are vulnerable to the shear stress and may lyse during cell culture as a result. In some embodiments, smaller vessels (e.g., flasks and/or beakers) are used, which may avoid wasting unnecessary media volumes that are otherwise used in a larger vessel. However, in such embodiments, cells cultured in the smaller vessels experience lysing with more frequency than in larger vessels.

Applicant has realized that by moving the liquid in the culture vessel via deformation of a portion of the vessel, various advantages may be realized. Accordingly, embodiments disclosed herein provide a vessel having a deformable portion that, in response to deformation, produces movement of the liquid sufficient to agitate and maintain the cells in suspension. In some embodiments, different deformation patterns (e.g., a wave of deformation) on the deformable portion are used to induce movement of the liquid in the vessel. In some embodiments, a shear stress applied to the cells as the liquid is moved via deformation of the deformable portion is reduced, which may minimize the frequency of lysing of the cells in the liquid.

According to one aspect, this document provides a vessel for culturing cells. In some embodiments, the vessel includes a vessel body arranged to hold a volume of liquid containing cells to be cultured, the vessel body having a deformable portion arranged to produce movement of the volume of liquid sufficient to maintain the cells in suspension. In some embodiments, the deformable portion is located on a base of the vessel, although the deformable portion may be located on another portion (e.g., a sidewall of the vessel).

In some embodiments, the pattern of deformation may include waves of deformation on the deformable portion of the vessel (e.g. a portion of the bottom of the vessel), although other patterns may be created to induce movement of the volume of liquid. For example, the deformation pattern may be circular in shape.

In some embodiments, the deformation is manually applied to the deformable portion of the vessel via a user. In other embodiments, the deformation is automated. For example, in some embodiments, the vessel may be placed on a deformation plate that has one or more moveable members configured to deform the deformable portion of the vessel.

In some embodiments, this document provides a vessel for culturing cells. In some embodiments, the vessel includes a vessel body arranged to hold a volume of liquid containing cells to be cultured, the vessel body having a base and a sidewall, at least a portion of the base being deformable. In response to a pattern of deformation in the base, the cells are maintained in suspension while minimizing a shear stress applied to the cells.

According to yet another embodiment, this document provides a system for culturing cells. In some embodiments, the system includes a vessel having a body arranged to hold a volume of liquid containing cells to be cultured, the vessel body having a deformable portion, and one or more moveable members configured to deform the deformable portion to produce a movement of the volume of liquid sufficient to maintain the cells in suspension.

In still another embodiment, this document provides a method of culturing cells. In some embodiments, the method includes placing a volume of liquid containing cells to be cultured in a vessel having a deformable portion, and deforming the deformable portion to produce a movement of the volume of liquid sufficient to maintain the cells in suspension.

In some embodiments, the deformable portion is formed integrally with or permanently attached to the vessel. In other embodiments the deformable portion may be remvoably attached to the vessel.

It should be appreciated that the foregoing concepts, and additional concepts discussed below, may be arranged in any suitable combination, as the present disclosure is not limited in this respect.

The foregoing and other aspects, embodiments, and features of the present teachings can be more fully understood from the following description in conjunction with the accompanying drawings.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. Various embodiments of the invention will now be described, by way of example, with reference to the accompanying drawings, in which:

FIGS. 1A and 1B are schematic illustrations of prior art vessels used for culturing cells;

FIG. 2A is a schematic illustration of a vessel for culturing cells according to one embodiment;

FIGS. 2B-2E are schematic illustrations of a base of the vessel of FIG. 2A with various arrangements of a deformable portion;

FIG. 3 is a schematic illustration of a liquid being mixed in a vessel via deformation in a deformable portion of the vessel via a moveable member;

FIG. 4 is a cross-sectional side view of the system of FIG. 3;

FIGS. 5A-5D are schematic illustrations of deformation patterns applied to a deformable portion;

FIG. 6 is a schematic of an illustrative embodiment of a cell culture incubator having an imager, a manipulator, and a vessel with a deformable portion on a manipulation location;

FIGS. 7-8 are schematics of illustrative embodiments of cell culture incubators; FIG. 7 shows a schematic of a cell culture incubator having a second imager; FIG. 8 shows a schematic of a cell culture incubator, wherein the imaging location and the manipulating location are the same location; and

FIG. 9 is a schematic depicting further components of cell culture incubators.

### DETAILED DESCRIPTION

Presently available vessels used to culture cells (e.g., spinner flasks and magnetic stirrers) rely on a rotating agitator to induce movement of the liquid. For example, the liquid may be rotated via an impeller or magnetic stirrer that agitates the cells and maintain the cells in suspension. In some embodiments, rotation of the agitator and liquid applies a shear stress on the cells. Unfortunately, cells may be vulnerable to such a shear stress, which, in such embodiments, may cause the cells to lyse. Such lysing also may be seen in greater frequency in smaller culture vessels than in larger culture vessels. In such embodiments, cell lysing may compromise the integrity of a cell culture and render the results useless.

Applicant has realized that by moving the liquid in the vessel via a deformation of a portion of the vessel, various advantages may be realized. As such, embodiments disclosed in instant document provide a vessel having a deformable portion that, in response to a deformation, induces movement in the liquid sufficient to maintain the cells in suspension. In some embodiments, different deformation patterns (e.g., a wave of deformation) may be applied to the deformable portion of the vessel to induce movement the liquid.

As used herein, a “deformable portion” of the vessel refers to a portion of the vessel that is capable of being at least partially deformed (e.g., moved) towards an interior of the vessel to induce movement of the liquid in the vessel. In some embodiments, the deformable portion is shape retaining such that after a deformation, the deformable portion returns to its original shape. As will be appreciated, in its original shape, the deformable portion may be planar and coterminous with non-deformed and/or non-deformable portion of the vessel. For example, in an embodiment in which at least a portion of the base is deformable, the original shape of the deformable portion is one that is coterminous with the rest of the base (e.g., with portions of the base that are non-deformable and/or portions that are deformable but have not yet been deformed). In some embodiments, the deformable portion is sufficiently rigid to maintain the shape of the culture vessel (e.g., in the shape of the beaker), while still allowing the deformable portion to be deformed.

In some embodiments, deformation of the deformable portion of the vessel may be manually applied (e.g., applied via a user's hand). In other embodiments, the deformation may be applied via moveable members of a deformation plate. For example, the vessel may be placed on a plate that has moveable members that may extend outwardly to from the plate deform the deformable portion of the vessel.

In some embodiments, the culture vessel with the deformable portion may be used in cell culture incubator having an incubator cabinet with an internal chamber for incubating cells in one or more cell culture vessels. In such embodiments, the culture vessel may be placed at a manipulator location (e.g., at a deformation plate), the manipulator location arranged to deform the deformable portion. In such embodiments, a manipulator (such as a pipette) may be used to transfer a volume of liquid into and out of the vessel during the cell culture. In some embodiments, the incubator cabinet also has a door opening from an external environment to the internal chamber. An imager may be used to image the cells within the internal chamber. A cell culture vessel transfer device may be used to move one or more cell culture vessels between locations within the internal chamber.

As used herein, an “incubator cabinet” is a housing that includes one or more chambers configured to hold one or more cell culture vessels. In some embodiments, an incubator cabinet includes a transfer chamber and an internal chamber, one or both of which are configured to hold one or more cell culture vessels. In some embodiments, an incubator may include one or more other elements such as one or more gas sources (e.g., a compressed gas cylinder or ozone generator), tubing (e.g., to convey one or more liquids or gases such as water, distilled water, deionized water, cell culture medium, air, carbon dioxide, ozone, and oxygen), airflow mechanisms (e.g., valves, release valves, pinholes, gas regulators, and mass flow regulators), pressure mechanisms (e.g., a pump such as a dry scroll pump, rotary pump, momentum transfer pump, diffusion pump, or diaphragm pump; a suction tube; a vacuum system; and an air blower), environmental monitors and controls (e.g., a gas sensor and/or monitor to sense and/or control concentrations of gases such as carbon dioxide, oxygen, and ozone; heat sources or sinks; temperature monitors and controls; humidity monitors; gas scrubbers; air filters; instrumentation for measuring particulate matter; pressure gauges; and flow meters), doors (e.g., openings or panels) windows (e.g., optical windows made of glass, plastic, composite, or other substantially transparent material for viewing an area inside the incubator cabinet), ports (e.g., to permit the introduction or removal of one or more gases or liquids), light sources (e.g., lamps, bulbs, lasers, and diodes), optical elements (e.g., microscope objectives, mirrors, lenses, filters, apertures, wave plates, windows, polarizers, fibers, beam splitters, and beam combiners), imaging elements (e.g., barcode readers, cameras, etc.), electrical elements (e.g., circuits, cables, power cords, and power supplies such as batteries, generators, and direct or alternating current supplies), computers, mechanical elements (e.g., motors, wheels, gears, robotic elements, and actuators such as pneumatic actuators, electromagnetic actuators, motors with cams, piezoelectric actuators, and motors with lead screws), and control elements (e.g., spin-wheels, buttons, keys, toggles, switches, cursors, screws, dials, screens, and touch-screens). In some embodiments, one or more of these other elements are part of the incubator, but are external to the incubator cabinet. In some embodiments, one or more of these other elements are included within the incubator cabinet.

As used herein, an “internal chamber” is a chamber disposed in an incubator cabinet. An internal chamber may include one or more windows (e.g., optical windows made of glass, plastic, composite, or other substantially transparent material for viewing an area inside the incubator cabinet). An internal chamber may include at least one door (e.g., for permitting the transfer of items into or out of the internal chamber). In some embodiments, the at least one door may be disposed between the internal chamber and a transfer chamber. In certain embodiments, an interlock may prevent the door from opening at an undesirable time (e.g., when a portion of the incubator cabinet is open to the surrounding environment so that contaminants cannot enter the internal chamber). An internal chamber may be of any appropriate size and geometry. In some embodiments, an incubator cabinet may include one or more partitions to define different regions of an internal chamber. One or more internal chambers or partitions thereof may have different environmental conditions. The environment (e.g., air pressure, gas content, temperature, light, and humidity) inside an internal chamber may be measured and/or controlled by one or more meters, monitors, sensors, controls, pumps, valves, apertures, and/or light sources. In some embodiments, an internal chamber may have a gas-tight or hermetic seal, e.g., around one or more windows or doors. In particular embodiments, sealants such as grease and/or mechanical elements such as o-rings, gaskets, septa, KF, LF, QF, quick coupling, or other sealing mechanisms may be used to establish one or more gas-tight seals. In some embodiments, grooves, depressions, protrusions, and/or molded plastic elements may facilitate in establishing one or more gas-tight seals.

An internal chamber may be made of any useful material. In some embodiments, an internal chamber may include one or more plastics, polymers, metals, or glasses.

As used herein, a “door” is an element that permits communication between two or more environments or regions when opened and prevents communication between the two or more environments or regions when closed. A door may be of any type, such as a sliding door, pocket door, swinging door, hinged door, revolving door, pivot door, or folding door. The door may be manually, mechanically, or electrically operated. For example, an operator may open or close a door by manually grasping, pulling, pushing, and/or otherwise physically interacting with the door or an element thereof (e.g., a handle) or by operating a mechanical control (e.g., a button, toggle, spin-wheel, key, switch, cursor, screw, dial, screen, or touch-screen). In certain embodiments, a door may be controlled by electrical or digital controls, such as by a computer. A door may be an automatically opening door. For example, a door may include a sensor, such as a pressure, infrared, motion, or remote sensor, that detects whether the door is open or closed and/or controls when the door opens or closes. A door may open by mechanical, pneumatic, electrical, or other means. In some embodiments, one or more doors may include one or more locking mechanisms. In particular environments, one or more doors may include one or more interlocks (e.g., a mechanical interlock such as a pin, bar, or lock or an electrical interlock such as a switch) to prevent one or more doors from opening at an undesirable time (e.g., when one or more chambers are open to the outside environment).

A transfer device for moving one or more items may be used to move items between the transfer chamber and the internal chamber. In some embodiments, the transfer device comprises a conveyor belt or other similar device for maneuvering items. Non-limiting examples of items that can be moved by transfer devices include cell culture vessels, pipettes, containers, syringes and other materials and instruments utilized in the culture of cells. In some embodiments, more than one transfer device may be included. In some embodiments, one or more transfer devices are located in the transfer chamber and/or in the internal chamber. In some embodiments, a transfer device may include one or more robotic elements. For example, a transfer device may comprise include one or more robotic arms capable of gripping, lifting, pushing, grabbing, sliding, rotating, translating, releasing, raising, lowering, and/or tilting one or more items (e.g., pipettes).

In some embodiments, the transfer device is a cell culture vessel transfer device. As used herein, a “cell culture vessel transfer device” refers to a device that can transfer one or more cell culture vessels from a first location to a second location. In some embodiments, the transfer device is anchored within the internal chamber. In certain embodiments, the transfer device may transfer one or more items to or from multiple locations in an incubator cabinet. For example, a cell culture vessel transfer device may be used to move a cell culture vessel from a transfer chamber to an internal chamber, and/or from a storage location to an imaging location. In some embodiments, an incubator cabinet includes more than one transfer device for moving one or more items (e.g., separate means for transferring items between and within chambers). A cell culture vessel transfer device may include one or more elements such as valves (e.g., electromagnetic or pneumatic valves), gears, motors (e.g., electrical or stepper motors), stages (e.g., xy or xyz stages), pistons, brakes, cables, ball-screw assemblies, rack-and-pinion arrangements, grippers, arms, pivot points, joints, translational elements, or other mechanical or electrical elements. In some embodiments, a cell culture vessel transfer device may include one or more robotic elements. For example, a cell culture vessel transfer device may include a robotic arm capable of gripping, lifting, pushing, grabbing, sliding, rotating, translating, releasing, raising, lowering, and/or tilting one or more cell culture vessels. In some cases, the cell culture vessel transfer device selectively and releasably grips one or more cell culture vessels. In certain embodiments, a cell culture vessel transfer device may include an arm coupled to a mechanical gripper. For example, an arm may include a mechanical gripper at or near one end for releasably gripping a cell culture vessel and be securely coupled to a surface or element of the incubator at or near the other end. In some embodiments, a robotic arm includes a pivot point where the mechanical gripper couples to the arm and one or more pivot and/or translational joints along the arm to permit flexible rotation and translation of a portion of the arm. In this manner, a robotic arm may access one or more cell culture vessels at different horizontal and vertical positions within an incubator cabinet (e.g., within a storage array in an internal chamber).

In some embodiments, a cell culture vessel transfer device is an automated transfer device. For example, the automated transfer device may be a robotic arm controlled by a computer that is programmed to move cell culture vessels from a storage location within the internal chamber of the incubator to an imaging location within the internal chamber of the incubator. In some embodiments, a cell culture vessel transfer device is manually operated. For example, a robotic arm located inside the internal chamber of an incubator may be operated by a user-controlled joystick from a location outside of the internal chamber of the incubator in order to move cell culture vessels from a storage location within the internal chamber of the incubator to an imaging location within the internal chamber of the incubator.

As used herein, a “cell culture vessel” is a device including a housing and one or more chambers for culturing cells. In some embodiments, the housing is a frame. The frame may be coupled to a lid. The one or more chambers may include cell culturing media including one or more membranes. In some embodiments, a cell culture vessel may include nutrients for promoting the growth of cells. In certain embodiments, a cell culture vessel may entirely enclose one or more cells or groups thereof. The housing of a cell culture vessel may include one or more pores or openings to permit the transfer of gases between a cell culture vessel and its surrounding environment. Non-limiting examples of cell culture vessels include flasks, suspension culture flasks, spinner flasks, plates, petri dishes and/or bags. In certain embodiments, a cell culture vessel includes a transparent or optically clear window. For example, a lid coupled to the housing of a cell culture vessel may include an optically clear portion for viewing cells e.g., with a microscope or other imager. In some embodiments, a cell culture vessel includes one or more portions that are substantially non-reflective. In some embodiments, the cell culture vessel is barcoded. Therefore, in some embodiments, the incubator includes a barcode reader.

In some embodiments, cell culture vessels may be pre-kitted with one or more reagents desired for a particular purpose, e.g., for growing cells, for differentiating cells, for subjecting cells to a particular assay condition, etc. In some embodiments, pre-kitted cell culture vessels contain reagents useful for performing a particular experiment (e.g., cell growth media, growth factors, selection agents, labeling agents, etc.) on a cell culture, in advance of the experiment. Pre-kitted cell culture vessels may facilitate experimental protocols by providing cell culture-ready vessels that do not require the addition of reagents. For example, progenitor cells from a patient may be added to a cell culture vessel pre-kitted with reagents for cell differentiation for the purpose of expanding a population of differentiated cells for autologous cell therapy. Pre-kitted cell culture vessels can be stored at any appropriate temperature, which is determined by the recommended storage parameters of the reagents within the pre-kitted cell culture vessel. In some embodiments, pre-kitted cell culture storage vessels are stored prior to use at temperatures between about −80° C. and about 37° C. In some embodiments, pre-kitted cell culture storage vessels are stored prior to use at temperatures between about −80° C. and about −20° C. In some embodiments, pre-kitted cell culture storage vessels are stored prior to use at temperatures between about −20° C. and about 4° C. In some embodiments, pre-kitted cell culture storage vessels are stored prior to use at temperatures between about 4° C. and about 37° C. In some embodiments, pre-kitted cell culture vessels are disposable. In some embodiments, pre-kitted cell culture vessels are reusable and/or refillable.

As used herein, a “storage location” refers to a location at which one or more cell culture vessels is stored (e.g., within an incubator cabinet). For example, one or more cell culture vessels may be stored at a storage location and later transferred to a different location (e.g., an imaging location). The storage location may be disposed in the internal chamber of the incubator cabinet. A storage location may be configured for storing a plurality of cell culture vessels. For example, a storage location may include one or more storage arrays, racks, shelves, pigeon-holes, cubbies, trays, slots, or other positions or mechanisms. In some embodiments, a storage location may be configured to store cell culture vessels horizontally, while in other embodiments it may configured to store cell culture vessels vertically. For example, a storage location may include a plurality of slots to receive cell culture vessels stacked vertically over one another. A storage location may be configured to hold 1, 2, 3, 4, 5, 6, 7, 8, 9, 10, 15, 20, 25, 30, 35, 40, 45, 50, 100, or any other number of cell culture vessels. In some embodiments, a storage location may be configured to hold greater than 100 cell culture vessels. In some embodiments, a storage location may include a mechanism for moving one or more storage arrays, racks, shelves, pigeon-holes, cubbies, trays, slots, or other positions or mechanisms. For example, a storage location may include one or more motors and movable stages (e.g., an xy or xyz stage) to move a storage rack from one position in an internal chamber to another position in an internal chamber, e.g., to facilitate access to one or more cell culture vessels stored in different locations. In some embodiments, the incubator cabinet may include one or more cell culture vessel transfer devices for moving one or more cell culture vessels.

A storage location may be configured to securely hold or receive one or more cell culture vessels. For example, one or more components of the storage location may include one or more locking mechanisms that have one or more adhesive, magnetic, electrical, and/or mechanical components (e.g., snaps, fasteners, locks, clasps, gaskets, o-rings, septa, springs, and other engagement members). In some embodiments, a storage location and/or cell culture vessel may include one or more grooves or depressions and/or may involve pieces of molded plastic. For example, a cell culture vessel may include one or more protruded features (e.g., a rim or knob) that are molded for insertion into one or more corresponding grooves, holes, or depressions at a storage location. In some cases, a cell culture vessel may include one or more grooves, holes, or depressions that are molded to fit one or more corresponding protruded features at a storage location.

As used herein, an “imager” refers to an imaging device for measuring light (e.g., transmitted or scattered light), color, morphology, or other detectable parameters such as a number of elements or a combination thereof. An imager may also be referred to as an imaging device. In certain embodiments, an imager includes one or more lenses, fibers, cameras (e.g., a charge-coupled device camera or CMOS camera), apertures, mirrors, light sources (e.g., a laser or lamp), or other optical elements. An imager may be a microscope. In some embodiments, the imager is a bright-field microscope. In other embodiments, the imager is a holographic imager or microscope. In other embodiments, the imager is a fluorescence imager or microscope.

As used herein, a “fluorescence microscope” refers to an imaging device which is able to detect light emitted from fluorescent markers present either within and/or on the surface of cells or other biological entities, said markers emitting light at a specific wavelength in response to the absorption a light of a different wavelength.

As used herein, a “bright-field microscope” is an imager that illuminates a sample and produces an image based on the light absorbed by the sample. Any appropriate bright-field microscope may be used in combination with an incubator cabinet provided herein.

As used herein, a “holographic imager” is an imager that provides information about an object (e.g., sample) by measuring both intensity and phase information of electromagnetic radiation (e.g., a wave front). For example, a holographic microscope measures both the light transmitted after passing through a sample as well as the interference pattern (e.g., phase information) obtained by combining the beam of light transmitted through the sample with a reference beam.

A holographic imager may also be a device that records, via one or more radiation detectors, the pattern of electromagnetic radiation from a substantially coherent source, diffracted or scattered directly by the objects to be imaged, without interfering with a separate reference beam and with or without any refractive or reflective optical elements between the substantially coherent source and the radiation detector(s).

In some embodiments, an incubator cabinet includes a single imager. In some embodiments, an incubator cabinet includes two imagers. In some embodiments, the two imagers are the same type of imager (e.g., two holographic imagers or two bright-field microscopes). In some embodiments, the first imager is a bright-field microscope and the second imager is a holographic imager. In some embodiments, an incubator cabinet comprises more than 2 imagers. In some embodiments, cell culture incubators comprise three imagers. In some embodiments, cell culture incubators having 3 imagers comprise a holographic microscope, a bright-field microscope, and a fluorescence microscope.

As used herein, an “imaging location” is the location where an imager images one or more cells. For example, an imaging location may be disposed above a light source and/or in vertical alignment with one or more optical elements (e.g., lens, apertures, mirrors, objectives, and light collectors).

As used herein, a “fiducial mark” refers to a feature that facilitates alignment of one or more components. In some embodiments, fiducial marks may include one or more hole apertures over a fluorescent media or printed or embossed fluorescent material. In other embodiments, fiducial marks may include grids, lines, or symbols. In some embodiments, one or more cell culture vessels include one or more fiducial marks to facilitate alignment of one or more cell culture vessels with an imager. In some embodiments, fiducial marks may be associated with moving parts, including transfer devices and robotics devices.

In some embodiments, a cell culture vessel is substantially aligned with an imager. In some embodiments, a cell culture vessel is substantially aligned with an imager via the use of at least one fiducial mark. As used herein, the term “substantially aligned” implies that one or more elements are substantially overlapping, identical, and/or in line with one another. The substantial alignment of one or more cell culture vessels at one or more locations (e.g., imaging locations) may facilitate the analysis of a sample by permitting overlapping images of the cell culture vessel to be obtained. For example, a cell culture vessel may be imaged at a first imaging location by a first imager and subsequently imaged at a second imaging location by a second imager. If the imaging fields of the respective imagers are substantially aligned, the images recorded by the first and second imagers may be combined (“stitched together”) for analysis. One or more fiducial marks present on one or more cell culture vessels may facilitate substantial alignment. In some cases, one or more fiducial marks present at one or more imaging or other locations (e.g., manipulation or maintenance locations) may facilitate substantial alignment.

As used herein, a “manipulator for manipulating cells” refers to a device for manipulating cells in the internal chamber. The manipulator may include one or more needles, capillaries, pipettes, and/or micromanipulators. For example, the manipulator may include a cell picker. A manipulator for manipulating cells may operate by detecting desirable cells or groups thereof present at a first location based on a predetermined criterion and transferring the desired cells or groups thereof from the first location to a second location. A cell picker may detect, pick, and/or transfer desirable or undesirable (e.g., pre-differentiated cell weeding) cells or groups thereof based on a manual or automated analysis. In some embodiments, information produced by an imager may be analyzed to detect desirable or undesirable cells. The cell picker may then transfer the desirable or undesirable cells to the second location. For example, an imager may image cells in or on a cell culture vessel at an imaging location, and the image used to identify desirable or undesirable cells or groups thereof. The cell picker may then transfer the desirable or undesirable cells, e.g., by contacting each desired cell or cells with a needle, capillary, pipette, or micromanipulator and effecting a movement of the cell or cells, from their first location to a second location in or on the cell culture vessel or elsewhere in the internal chamber. In some embodiments, the first location of the cells may be in or on a cell culture vessel. In particular embodiments, a cell picker transfers cells from a first location in or on a cell culture vessel to a second location on the same cell culture vessel. In other embodiments, a cell picker transfers cells from a first location in or on a first cell culture vessel to a second location in or on a second cell culture vessel. In certain other embodiments, a cell picker transfers cells from a first location in or on a cell culture vessel to a second location in the internal chamber that is not in or on a cell culture vessel.

In some embodiments, the manipulator includes at least one microelectrode. As used herein, the term “microelectrode” refers to an electrical conductor used to deliver electrical stimulation to a cell. For example, microelectrodes can be used to deliver genetic material into a cell by electroporation. In some embodiments, the manipulator includes at least one microinjector. Generally, microinjectors are glass micropipettes that have been pulled to form a sharp, hollow structure capable of piercing the membrane of a cell and serving as a conduit for the introduction of genetic material into the cell.

In some embodiments, a manipulator is manually operated. For example, a manipulator having a cell picker located inside the internal chamber of an incubator cabinet may be electronically-linked to and controlled by a user-directed joystick located outside the internal chamber of the incubator cabinet. In some embodiments, the user-directed joystick is connected to a display device. In some embodiments, the display device shows images captured by an imaging device inside the internal chamber of the incubator cabinet.

In some embodiments, a manipulator is automated. For example, a manipulator inside an internal chamber of an incubator cabinet may be electronically connected to a controller outside of the incubator cabinet, which is electronically connected to a computer that directs the manipulator. For example, the controller may instruct the manipulator to apply a vacuum to one or more pipette tips fluidly connected to the manipulator.

One or more elements of the manipulator for manipulating cells may be sterilized, for example using a sterilizing composition or method (e.g., ethanol or ozone gas, UV Light, Hydrogen peroxide), prior to manipulation.

As used herein, “manipulation location” refers to the location at which cells are manipulated by a manipulator for manipulating cells (e.g., a cell picker). In certain embodiments, the manipulation location may be the same as the imaging location.

According to one aspect, the cell culture incubator includes an incubator cabinet with an imaging location and a manipulating location. Cells of a cell culture vessel are imaged at the imaging location by an imager and manipulated at the manipulating location by a manipulator. In some embodiments, the imaging location and the manipulating location are two distinct locations within the incubator cabinet. The cell culture incubator may include a transfer device that moves cell culture vessels between the imaging location and the storage location. In other embodiments, the imaging location and the manipulating location are the same, such that the cells of culture vessels are imaged at the manipulation location.

In some embodiments, an imager may be used in conjunction with a manipulator. For example, an imager may image cells in or on a cell culture vessel at an imaging location, and the image used to identify desirable cells or groups thereof. The cell picker, which may or may not be resident at the imaging location, may then transfer the desirable or undesirable cells, e.g., by contacting each desired cell or cells with a needle, capillary, pipette, or micromanipulator and effecting a movement of the cell or cells, from their first location to a second location in or on the cell culture vessel or elsewhere in the internal chamber. In some embodiments, a cell picker transfers cells from a first location in or on a cell culture vessel to a second location on the same cell culture vessel. In other embodiments, a cell picker transfers cells from a first location in or on a first cell culture vessel to a second location in or on a second cell culture vessel. In certain other embodiments, a cell picker transfers cells from a first location in or on a cell culture vessel to a second location in the internal chamber that is not in or on a cell culture vessel.

In some embodiments, a single location within the incubator cabinet may serve as an imaging location and a manipulating location. In one embodiment, cells are imaged as they are manipulated by the manipulator. In some embodiments, the imaging location and imaging location may be at separate locations within the incubator cabinet.

In some embodiments, the manipulator includes sensors that allow it to report its position and determine when it has touched the bottom of the cell culture vessel. In some embodiments, an imager may be used to guide the manipulator in order to achieve repeatability and accuracy. In some embodiments, compliance (e.g., springiness) in the manipulator may be used to relax the need for extreme mechanical accuracy.

In some embodiments, one or more manipulators are used to manipulate one or more pipettes as described in this document. In some embodiments, manipulators are used to place and/or remove pipette tips on the pipettes.

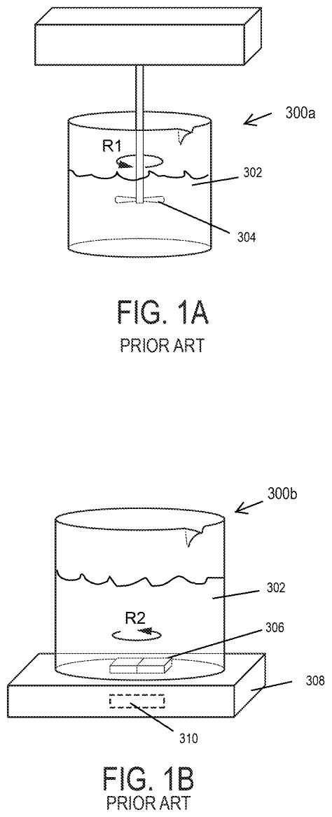

Turning now to the figures, FIGS. 1A and 1B illustrate culture vessels according to the prior art. As shown in FIG. 1A, in one example, the culture vessel is a spinner flask (300a) that induces movement of a liquid (302) via an impeller (304) that is rotated (see arrow labeled R1) near a center of the liquid. In some embodiments, the impeller (304) may be rotated via a gear-driven motor (not shown). As shown in FIG. 1B, in another example, the culture vessel includes a magnetic stirrer (300b) that induces movement of the liquid (302) via rotation of a magnetic stir bar (306) (see arrow labeled R2) resting on a bottom of the vessel. As will be appreciated, the magnetic stir bar may be rotated via a rotating magnetic field generated by either a rotating magnet 310 or a set of stationary electromagnets (not shown) in a stir plate 308.

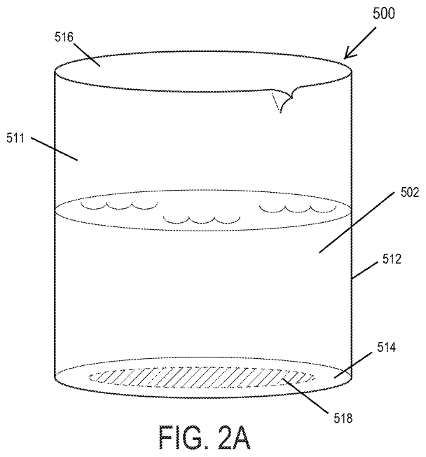

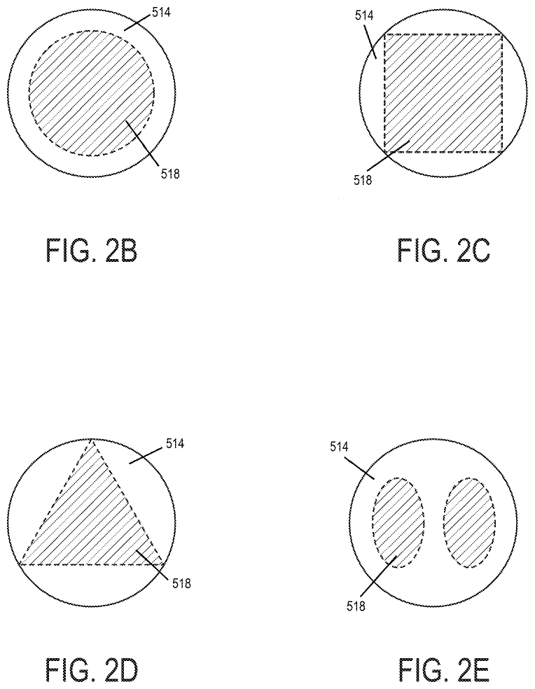

FIG. 2A illustrates a culture vessel (500) according to the present document, with a deformable portion arranged to induce movement of a volume liquid in the vessel. As shown in FIG. 2, in one embodiment, the vessel (500) has a body (511) with a sidewall (512), a base (514) and an open top (516). In such embodiment, the vessel body (511) is arranged to hold a volume of liquid (502) with cells. In some embodiments, a lid (not shown) may be placed on the vessel body (511) to cover the open top (516). As will be appreciated, although the vessel in FIG. 2 has a cylindrically shaped body (511), other shaped vessels may be used in other embodiments.

In some embodiments, at least a portion of the vessel body (511) is deformable. For example, as shown in FIG. 2A, a portion (518) of the base (514) may be deformable. In some embodiments, the deformable portion (518) may be located in an inner area of the base (514), although the deformable portion (518) also may be located in another suitable area of the base (514). In some embodiments, the entire base (514) is configured to be deformable.

In some embodiments, as shown in FIGS. 2A and 2B, the deformable portion (518) may be circular in shape. The deformable portion also may be square or triangular in shape, as shown in FIGS. 2C and 2D, or have another suitable shape (e.g., rectangular, ovular), as will be appreciated.

Although only one deformable portion is shown in FIG. 2A, the base (514) may have two or more deformable portions (518) in other embodiments. For example, as illustrated in FIG. 2E, in some embodiments, the base (514) may have two deformable portions (518). As will be appreciated, in such embodiments, the two or more deformable portions need not be the same size. That is, a first deformable portion may be larger than a second deformable portion. The two or more deformable portions also need not have the same shape. For example, a first deformable portion may be rectangular while a second deformable portion may be circular.

Although the vessel body is shown with only a portion of the base being deformable, it will be appreciated that other portions of the vessel body may be deformable. For example, a portion of the sidewall, or two or more portions of the sidewall, may be deformable. In some embodiments, the entire side wall may be deformable.

In other embodiments, the vessel body may include deformable portions (e.g., one or more deformable portions) on both the sidewall and the base. In such embodiments, the sidewall and base need not include deformable portions having the same number, size, or shape. For example, while the entire base may be deformable, the sidewall may include only one, smaller, square-shaped deformable portion

In some embodiments, the deformable portion is made of a flexible material, such as a plastic or elastomeric material or a polymer. As will be appreciated, other suitable materials also may be used. In some embodiments, the type, thickness and/or properties of the material(s) used to form the deformable portion may be the same or may vary from deformable portion to deformable portion. For example, a deformable portion may have a thinner inner area than that of an outer area. Such a deformable portion also may be made of a material (e.g., a plastic) with a smaller Young's modules (e.g., be more subject to deformation) in the inner area than that of the outer area. In other embodiments, a first deformable member may be formed of a first material having a first thickness and a second deformable member may be formed of the first material having a second thickness, the first and second thicknesses being different.

As will be appreciated, the size, shape, number, and material used to form the deformable portion(s) may be used to tailor the intensity and location of the deformation applied to the liquid. In such embodiments, this may affect the intensity and direction of fluid movement. For example, a deformable portion that is more flexible may allow a more forceful deformation to be made to induce fluid flow. In another example, if a horizontal direction of fluid flow is desired, a deformable portion may be positioned on a sidewall of the vessel body. In some embodiments, more deformable portions in the side wall may allow for more deformations to induce fluid flow in the horizontal direction. In a similar fashion, a larger deformable portion may allow for one larger deformation and/or for several smaller deformations to induce such a fluid flow.

In some embodiments, the deformable portion is integrally formed with the vessel body. For example, the vessel body may be comprised of a single plastic material, with the deformable portion being made thinner and more malleable than the rest of the vessel body. The deformable portion also may be made of a different material than the rest of the vessel body, with the deformable portion being attached thereto (e.g., via glue, welding, heat sealing, etc.).

In other embodiments, the deformable portion may be removably attached to the vessel body. For example, the base of the vessel body may include an opening into which the deformable portion is snapped or threaded thereto. In other embodiments, the base of the vessel body may be separable from the side wall, such that a base or sidewall with one or more deformable portions may be attached to a corresponding sidewall or base, respectively (e.g., which may or may not have deformable portions).

In some embodiments, a kit having a sidewall and several different bases may be prepare, with each base being formed with different shapes, sizes and numbers of and/or materials used to form the deformable portions. As will be appreciated, the user may select the base with the desired number and/or shape of deformable portions and may thereafter attach (e.g., threadably attach) the base to the sidewall. The user also may select the base with the desire material used to form the deformable portion and attach the base to the sidewall. In such embodiments, the user may choose the base that will deliver the desired level of deformation (and fluid flow movement), and may attach such a base to the sidewall for a cell culture.

Although the kit has been described with one sidewall and various bases, in other embodiments, the kit may have one base and several sidewalls with different deformable portions to select from and attach to the base for a cell culture. In another embodiment, the kit may have several bases and several kits, each having different deformable portions to select and attach to one another.

According to one aspect, the deformable portion is arranged to induce movement of the volume of liquid sufficient to maintain cells in suspension. As used herein, maintaining cells in suspension refers to cells that are moving within the liquid. That is, the cells have not fallen to the base of the vessel body. As will be appreciated, in such embodiments, the volume of liquid in the vessel body is moved with a velocity sufficient to agitate the cells so that the cells are no longer resting on the base of the vessel body.

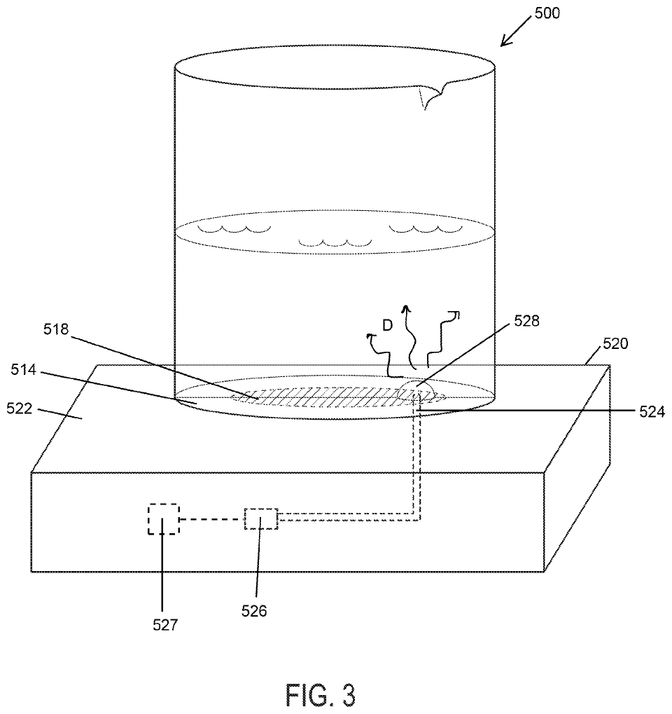

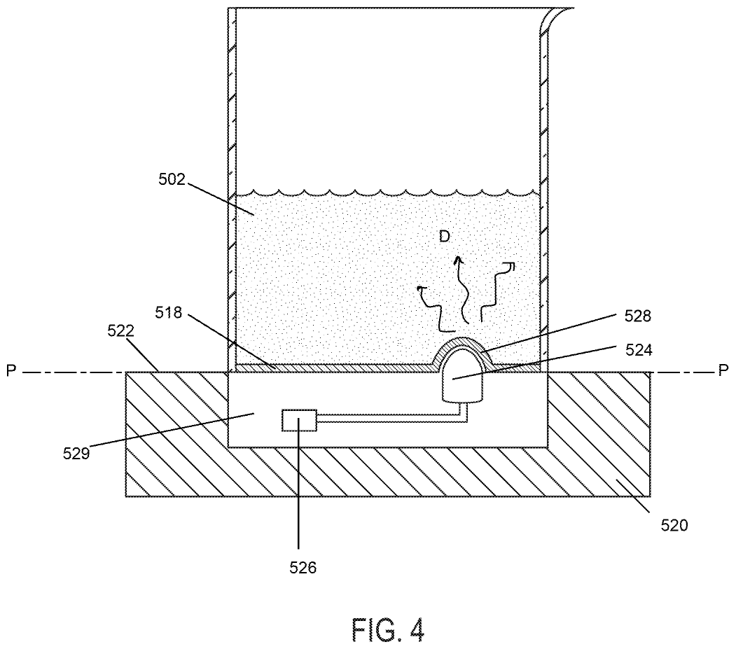

According to another aspect, the deformable portion may be automatically deformed, such as via deformation plate (520), to produce liquid movement. For example, as shown in FIGS. 3 and 4, the base (514) of the vessel (500) may be placed on a top surface (522) of the deformation plate (520), the deformation plate (520) having a moveable member (524) moveable by an actuator (526). Once the deformation plate (520) is engaged (e.g., turned on), the moveable member (524) may move to a forward position, beyond the top surface (522), to contact and deforms the deformable member (514). As will be appreciated, the top surface (522) may be formed of a flexible material (e.g., an elastomeric material) that allows the moveable members to extend outwardly therefrom. In some embodiments, the top surface (522) is formed of a material that is shape retaining, such that the top surface returns to its original configuration.

FIGS. 3 and 4 illustrate a deformation (528) of the deformable portion 518 that has been produced via the moveable member (524) of the deformation plate (520). As shown in these figures, the deformation (528) extends towards an interior of the vessel body (511). As also shown in these figures, the deformation causes a movement of liquid, also towards the interior of the vessel body (511), as illustrated by the arrows labeled D. For example, the deformation (528) may cause a ripple effect in the liquid.

Although only one moveable member is shown in FIGS. 3 and 4, it will be appreciated that the deformation plate may have two or more moveable members in other embodiments. In such embodiments, the moveable members may be attached to and actuated by the same actuator, although each moveable member also may have its own corresponding actuator. As will also be appreciated, the moveable member may be directly or indirectly connected to the actuator.

Although the vessel is shown on top of a deformation plate with moveable members that extend outwardly (e.g., in a vertical direction) to deform the deformable portion on the base, in embodiment in which the deformable portion is locate in the sidewall, the vessel may be placed adjacent a housing with moveable members that extend outwardly (e.g., in a horizontal direction) to deform the deformable portion on the sidewall.

Turning back to FIG. 3, the actuator (526) is operatively connected to a controller, which instructs the actuator to move the moveable member (527). For example, in response to a user command (e.g., pressing an start button on the deformation plate, the controller may instructs actuator to move the moveable member between forward and retracted positions. As will be appreciated, in the forward position, the actuator may extend outwardly beyond a plane P-P passing through the top surface (522) of the deformation plate (520). In the retracted position, the moveable member (528) remains below the top surface (522), in a housing (529) of the deformation plate.

In some embodiments, the controller (527) instructs the actuator (526) to move the moveable member (524) and deform the deformable portion only once. In such an embodiment, the user must continue to provide user commands (e.g., pressing a start button on the plate) to continue to move the moveable members. In other embodiments, in response to a user command, the controller (527) may instruct the actuator (526) to carry out a program, in which a sequence of deformations take place.

In some embodiments, the program may set a period of time for deforming the deformable portion (e.g., for three hours) or a set number of deformations to perform (e.g., 1000 deformations). In other embodiments, the program may set a frequency of deformations (e.g., 10 deformations per minute) and/or the intensity of the deformations (e.g., strong). For example, the user may control the strength at which the deformable portion is deformed, and, thus, the cells are agitated. As will be appreciated, with respect to the intensity and frequency, the program may instruct the actuator to deform the deformable portion at the same strength and frequency for the duration of the program. However, the intensity and frequency of deformations may be changed depending upon the desired liquid movement. For example, the deformations may start of slow and infrequent and become stronger and more frequent.

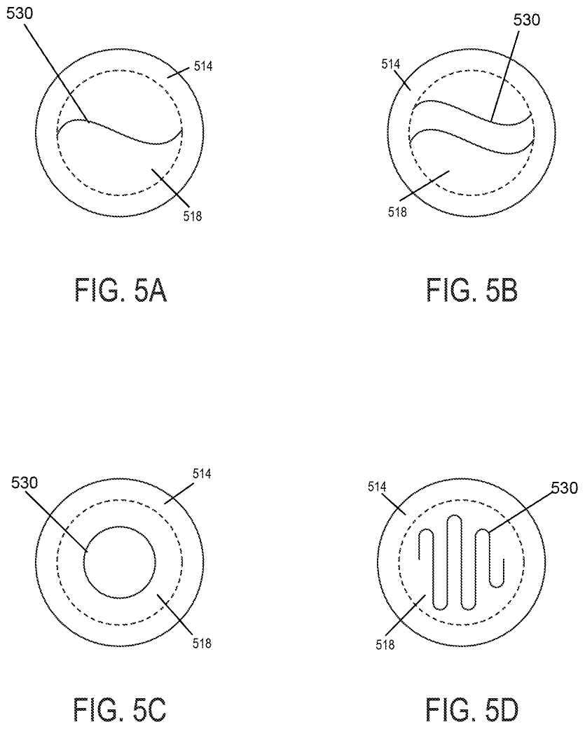

As shown in FIG. 4, in one embodiment, the moveable member may deform the deformable portion (518) by repeatedly moving between the forward and retracted positions at the same location of the deformable portion (518). In other embodiments, once the moveable member is in the forward position, the actuator (526) may move the moveable member (524) in different directions across the deformable portion (518). That is, the moveable members may create a pattern of deformation (530) across the deformation portion (518). For example, as shown in FIG. 5A, in some embodiments, the moveable member (524) may be moved so as to create a wavelike deformation pattern (530). As shown in FIG. 5C, in other embodiments, the moveable member (524) may be rotated to create a circular deformation pattern (530). The moveable member (524) also may be moved so as to create a serpentine deformation pattern (530), as shown in FIG. 5D. As will be appreciated, other deformation patterns may be used in other embodiments. As will be further appreciated, the deformation pattern may be chosen to create certain directions of flow (e.g., flow profiles) in the liquid for agitating the cells.

In embodiments having two or more moveable members, each moveable member may create a deformation pattern (530) across the deformable member. In some embodiments, the moveable members may create the same deformation pattern (530) across the deformable member (518). For example, as shown in in FIG. 5B, two wavelike deformation patterns have been created. As will be appreciated, the deformation patterns created by first and second moveable members need not be the same in other embodiments.

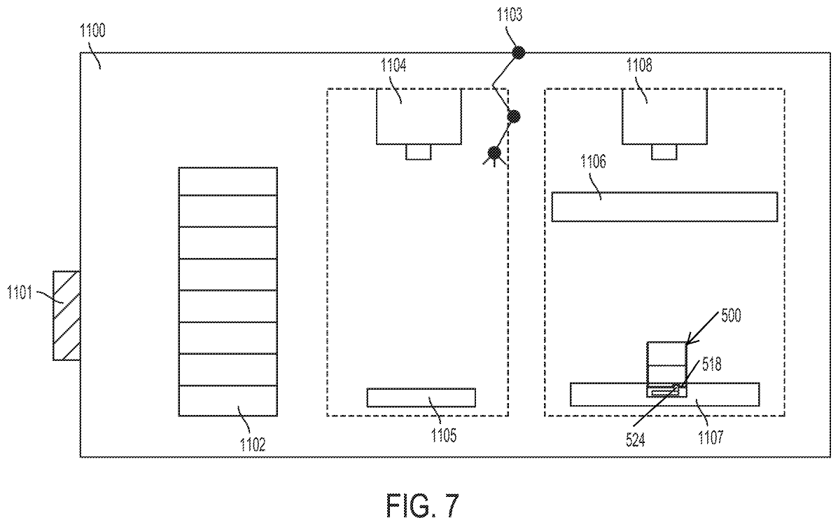

FIG. 7 illustrates an embodiment in which the vessel (500) with one or more deformable portions (518) is used in a cell culture incubator. The cell culture incubator includes an incubator cabinet having an internal chamber (1100) for incubation of cells in one or more cell culture vessels, such as vessel (500). The incubator cabinet includes an external door (1101) that opens and closes to permit communication between an external environment and the incubator cabinet. In some embodiments, the external door opens and closes to permit communication between an external environment and the internal chamber. The internal chamber is configured to hold one or more cell culture vessels, such as the vessel (500). The one or more cell culture vessels are stored in a storage location (1102). In some embodiments, the storage location is a free-standing structure. For example, a storage location may be a test tube or culture flask rack that can be removed from the internal chamber of the incubator for loading and unloading of culture vessels. In some embodiments, the storage location is affixed to a surface of the internal chamber. For example, the storage location may be a series of racks or shelves that are connected to the walls or floor of the internal chamber and are thus not able to be removed from the incubator cabinet.

In some embodiments, the cell culture incubator includes a cell culture vessel transfer device (1103) for moving one or more cell culture vessels. The cell culture transfer device may be affixed to any appropriate surface of the internal chamber of the incubator. For example, the cell culture vessel transfer device may be affixed to the top or ceiling of the internal chamber. Alternatively, the cell culture vessel transfer device may be affixed to a side wall of the internal chamber. In some embodiments, the cell culture vessel transfer device is not affixed to the wall of the internal chamber. For example, the cell culture vessel transfer device may rest on a wheeled tripod or other mobile structure that can be moved around the internal chamber.

In some embodiments, the transfer device moves one or more cell culture vessels from a storage location (1102) to an imaging location (1105) or to a manipulation location (1107). The transfer device (1103) can also move one or more cell culture vessels from an imaging location (1105) to a manipulation location (1107) or from a manipulation location (1107) to an imaging location (1105). When imaging or manipulation are complete, the transfer device (1103) moves one or more cell culture vessels from an imaging location (1105) or a manipulation location (1107) to a storage location (1102).

In some embodiments, the incubator cabinet includes a first imaging location (1105) and a manipulation location (1107). In some embodiments, one or more imaging locations are located on a surface of the internal chamber opposite from an imager. In some embodiments, imaging locations are platforms, either free-standing or affixed to a surface of the internal chamber. In some embodiments, the platform is movable. For example, a movable platform may be affixed to two or more rods that allow the platform to be moved left, right, forward, backward, up or down in relation to an imager. In some embodiments, the movable platform is motorized.

In some embodiments, the incubator cabinet includes a first imager (1104) that images the cells of cell culture vessels when the vessels are at the first imaging location (1105). In some embodiments, the first imager is a bright-field microscope. In some embodiments, the first imager is a holographic microscope.

In some embodiments, a manipulator (1106) manipulates the cells of cell culture vessels when the vessels are at the manipulation location (1107). In some embodiments, the manipulator has an array of needles, capillaries, pipettes, and/or micromanipulators. For example, the manipulator may include a cell picker. In some embodiments, a manipulator has a cell picker. Generally, manipulation locations share many characteristics with imaging locations, as described herein.

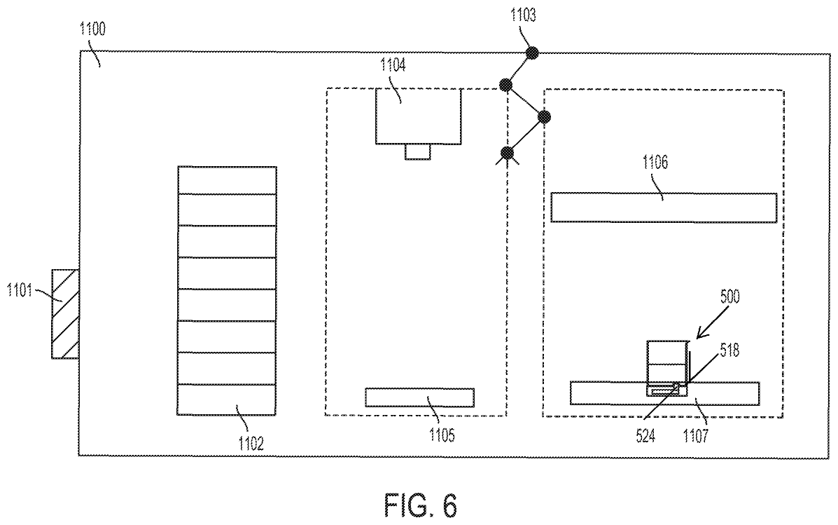

As shown in FIG. 6, in one embodiment, the vessel (500) with a deformable portion (518), may be placed on the manipulation location (1107) to mix the liquid (502) with cells. In such an embodiment, the manipulation location (1107) may include a deformation plate, as described herein, with one or more moveable members (524) that deform the deformable portion (518) (e.g., according to a prescribed deformation pattern (530).

FIG. 7 depicts another illustrative embodiment of a cell culture incubator. In some embodiments, the incubator cabinet has a second imager (1108). The second imaging location may be at or near the manipulation location (1107). In some embodiments, the second imaging location and the manipulation location (1107) are the same location. In some embodiments, a second imager (1108) images the cells of cell culture vessels while the cells are manipulated by the manipulator (1106). In some embodiments, the second imager is a bright-field microscope. In some embodiments, the second imager is a holographic microscope.

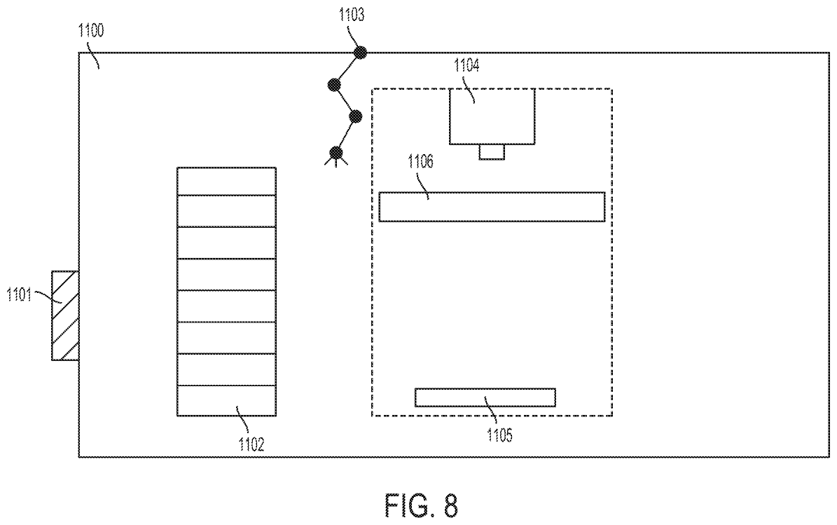

FIG. 8 depicts one illustrative embodiment of a cell culture incubator. In some embodiments, the cell culture incubator has an imaging location and a manipulation location that are the same location (1105).

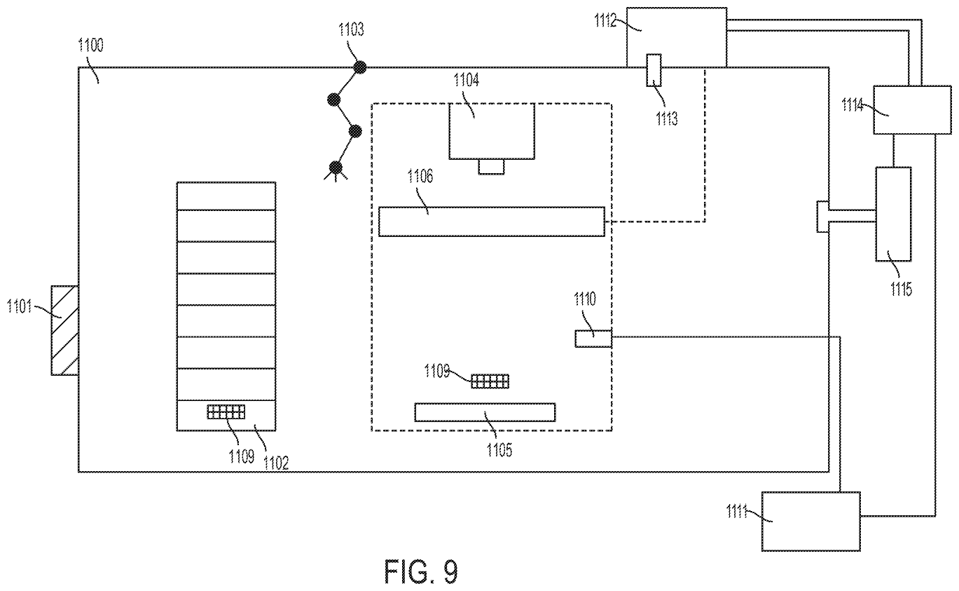

FIG. 9 depicts illustrative embodiments of further components of a cell culture incubator. As will be appreciated, further components are any component of the incubator that is not otherwise listed or show in one of the previous embodiments or figures. In some embodiments, a cell culture incubator contains barcoded cell culture vessels (1109). Thus, in some embodiments, a cell culture incubator has a barcode scanner (1110) located inside the internal chamber of the incubator cabinet. In some embodiments, the barcode reader communicates with a computer (1111) to relay information about the cell culture vessel for which the barcode has been scanned. In some cases, a barcode scanner may be affixed to any surface of the internal chamber. For example, a barcode scanner can be affixed to a wall of the internal chamber in close proximity to an imaging location (1105).

In some embodiments, the cell culture incubator contains at least one probe and/or at least one sensor (1113) that measures environmental conditions within the internal chamber. Examples of probes to measure environmental conditions include but are not limited to temperature probes, pressure probes, carbon dioxide (CO₂) sensors, oxygen (O₂) sensors and relative humidity sensors. In some embodiments, the at least one probe and/or at least one sensor located within an instrument housing (1112). The at least one probe and/or at least one sensor is connected to a controller (1114). In some embodiments, the controller (1114) communicates with a computer (1111). Additionally, the controller (1114) may communicate with a fluid dispensing system (1115). For example, if a CO₂ sensor indicates a low CO₂ level in the internal chamber, the controller (1114) may direct the fluid dispensing system (1116) to inject CO₂ gas into the internal chamber in order to increase the CO₂ level of the internal chamber.

As will be appreciated, the controller (1114) also may communicate with the manipulation location (1107) to move one or more moveable members (524) to deform the deformable portion (518) of the vessel (e.g., the vessel base (514)) to mix the liquid (502) in the vessel. In such an embodiment, the controller may instruct the agitator to move the one or more moveable members according to a program.

### Automated Cell Culture

This document relates to incubators and methods for culturing, manipulating, and/or monitoring cells under controlled conditions (e.g., under aseptic and/or sterile conditions). In some aspects, incubators and methods include automated components. In some aspects, incubators and methods are useful for long term cell culture (e.g., to grow and maintain cells for recombinant protein expression or to grow and/or differentiate cells for therapeutic applications such as implantation). In some embodiments, cell cultures are grown within a culture vessel, such as vessel (500), in an incubator described herein. Mixing of the liquid in such embodiments may be performed by deforming the deformable portion (518) of the vessel body (514).

### Culture Vessel

Cell culture vessels, such as vessel (500), may be configured for culturing cells of different types, including eukaryotic or prokaryotic cells. In some embodiments, cells are mammalian cells (e.g., human cells, canine cells, bovine cells, ovine cells, feline cells, or rodent cells such as rabbit, mouse, or rat cells). In some embodiments, cells are insect cells, avian cells, microbial cells (e.g., yeast cells such as Saccharomyces cerevisiae, Kluyveromyces lactis, or Pischia pastoris cells, or bacterial cells such as Escherichia coli, Bacillus subtilis, or Corynebacterium cells), insect cells (e.g., Drosophila cells, or Sf9 or Sf21 cells), plant cells (e.g., algal cells) or cells of any other type.

In some embodiments, cells are cultured for producing natural products (e.g., taxols, pigments, fatty acids, biofuels, etc.). In some embodiments, cells are cultured to express recombinant products (e.g., recombinant protein products such as antibodies, hormones, growth factors, or other therapeutic peptides or proteins). In some embodiments, cells are expanded and/or differentiated for therapeutic use such as implantation into a subject (e.g., a human subject) in order to provide or supplement a cellular, tissue, or organ function that is missing or defective in the subject.

In some embodiments, cells are from immortalized cell lines. Non-limiting examples of cell lines include human cells, for example, HeLa cells, prostate cancer cells (e.g., DU145, PC3 and/or Lncap cells), breast cancer cells (e.g., MCF-7, MDA-MB-438, and/or T47D cells), acute myeloid leukemia cells (e.g., THP-1 cells), glioblastoma cells (e.g., U87 cells), neuroblastoma cells (e.g., SHSY5Y cells), bone cancer cells (e.g., Saos-2 cells) and chronic myelogenous leukemia cells (e.g., KBM-7 cells). In some embodiments, cell lines include primate cell lines, rodent cell lines (e.g., rat or mouse cell lines), canine cell lines, feline cell lines, Zebrafish cell lines, Xenopus cell lines, plant cell lines, or any other cell. In some embodiments, cells are human 293 cells (e.g., 293-T or HEK 293 cells), murine 3T3 cells, Chinese hamster ovary (CHO) cells, CML T1 cells, or Jurkat cells.

In some embodiments, cells are primary cells, feeder cells, or stem cells. In some embodiments, cells are isolated from a subject (e.g., a human subject). In some embodiments, cells are primary cells isolated from a tissue or a biopsy sample. In some embodiments, cells are hematopoietic cells. In some embodiments, cells are stem cells, e.g., embryonic stem cells, mesenchymal stem cells, cancer stem cells, etc. In some embodiments, cells are isolated from a tissue or organ (e.g., a human tissue or organ), including but not limited to, solid tissues and organs. In some embodiments, cells can be isolated from placenta, umbilical cord, bone marrow, liver, blood, including cord blood, or any other suitable tissue. In some embodiments, patient-specific cells are isolated from a patient for culture (e.g., for cell expansion and optionally differentiation) and subsequent re-implantation into the same patient or into a different patient. Accordingly, in some embodiments, cells grown in the incubators described herein may be used for allogenic or autogeneic therapy. In some embodiments, cells grown in the incubators disclosed herein may be genetically modified, expanded and reintroduced into a patient for the purpose of providing an immunotherapy (e.g., chimeric antigen receptor therapy (CAR-T), or delivery of CRISPR/Cas modified cells).

In some embodiments, a primary cell culture includes epithelial cells (e.g., corneal epithelial cells, mammary epithelial cells, etc.), fibroblasts, myoblasts (e.g., human skeletal myoblasts), keratinocytes, endothelial cells (e.g., microvascular endothelial cells), neural cells, smooth muscle cells, hematopoietic cells, placental cells, or a combination of two or more thereof.

In some embodiments, cells are recombinant cells (e.g., hybridoma cells or cells that express one or more recombinant products). In some embodiments, cells are infected with one or more viruses.

### Primary Cell Isolation

In some embodiments, cells are isolated from tissues or biological samples for ex vivo culture in the incubators provided herein. In some embodiments, cells (e.g., white blood cells) are isolated from blood. In some embodiments, cells are released from tissues or biological samples using physical and/or enzymatic disruption. In some embodiments, one or more enzymes such as collagenase, trypsin, or pronase are used to digest the extracellular matrix. In some embodiments, tissue or biological samples are placed in culture medium (e.g., with or without physical or enzymatic disruption), and cells that are released and that grow in the culture medium can be isolated for further culture.

### Cell Culture

As used herein, cell culture refers to a procedure for maintaining and/or growing cells under controlled conditions (e.g., ex vivo). In some embodiments, cells are cultured under conditions to promote cell growth and replication, conditions to promote expression of a recombinant product, conditions to promote differentiation (e.g., into one or more tissue specific cell types), or a combination of two or more thereof.

In some embodiments, cell culture vessels are configured for culturing cells in suspension. In some embodiments, cell culture vessels are configured for culturing adherent cells. In some embodiments, cell culture vessels are configured for 2D or 3D cell culture. In some embodiments, cell culture vessels include one or more surfaces or micro-carriers to support cell growth. In some embodiments, these are coated with extracellular matrix components (e.g., collagen, fibrin and/or laminin components) to increase adhesion properties and provide other signals needed for growth and differentiation. In some embodiments, cell culture vessels include one or more synthetic hydrogels such as polyacrylamide or polyethylene glycol (PEG) gels to support cell growth. In some embodiments, cell culture vessels include a solid support with embedded nutrients (e.g., a gel or agar, for example for certain bacterial or yeast cultures). In some embodiments, cell culture vessels include a liquid culture medium.

In some embodiments, cells are cultured in one of any suitable culture media. Different culture media having different ranges of pH, glucose concentration, growth factors, and other supplements can be used for different cell types or for different applications. In some embodiments, custom cell culture media or commercially available cell culture media such as Dulbecco's Modified Eagle Medium, Minimum Essential Medium, RPMI medium, HA or HAT medium, or other media available from Life Technologies or other commercial sources can be used. In some embodiments, cell culture media include serum (e.g., fetal bovine serum, bovine calf serum, equine serum, porcine serum, or other serum). In some embodiments, cell culture media are serum-free. In some embodiments, cell culture media include human platelet lysate (hPL). In some embodiments, cell culture media include one or more antibiotics (e.g., actinomycin D, ampicillin, carbenicillin, cefotaxime, fosmidomycin, gentamycin, kanamycin, neomycin, penicillin, penicillin streptomycin, polymyxin B, streptomycin, tetracycline, or any other suitable antibiotic or any combination of two or more thereof). In some embodiments, cell culture media include one or more salts (e.g., balanced salts, calcium chloride, sodium chloride, potassium chloride, magnesium chloride, etc.). In some embodiments, cell culture media include sodium bicarbonate. In some embodiments, cell culture media include one or more buffers (e.g., HEPES or other suitable buffer). In some embodiments, one or more supplements are included. Non-limiting examples of supplements include reducing agents (e.g., 2-mercaptoethanol), amino acids, cholesterol supplements, vitamins, transferrin, surfactants (e.g., non-ionic surfactants), CHO supplements, primary cell supplements, yeast solutions, or any combination of two or more thereof. In some embodiments, one or more growth or differentiation factors are added to cell culture media. Growth or differentiation factors (e.g., WNT-family proteins, BMP-family proteins, IGF-family proteins, etc.) can be added individually or in combination, e.g., as a differentiation cocktail comprising different factors that bring about differentiation toward a particular lineage. Growth or differentiation factors and other aspects of a liquid media can be added using automated liquid handlers integrated within the incubators.

In some aspects, devices and methods described herein provide and maintain appropriate temperature and gas mixtures for cell growth. It should be appreciated that cell growth conditions differ for different cell types and that devices described herein can be programmed to maintain different conditions. In some embodiments, conditions of approximately 37° C., and 5% CO₂ are used for mammalian cells.

In some embodiments (e.g., for adherent cell cultures), culture media can be removed directly by aspiration and replaced with fresh media. In some embodiments (e.g., for non-adherent/suspension cultures), media changes can involve centrifuging a cell culture, removing the old culture media and replacing it with fresh media. In some embodiments, the centrifuge is located in the internal chamber of an incubator. In some embodiments, culture vessels allow for continuous media replacement. In some embodiments, the incubators described herein may include one or more components that can be used to process, replace, supply, and/or maintain different aspects of a culture media to support cells. Incubators may include a reservoir containing waste media and/or a reservoir containing fresh media. Such reservoirs may be present (e.g., for temporary storage) within a refrigerator inside the incubator or a refrigerated section of the incubator. In some embodiments, one or more reservoirs are provided outside the incubators and piping is provided into and out from the incubator space to supply or draw from a liquid handler units (e.g., liquid handle units having an aspirator) or temporary reservoir within the incubator to facilitate cells feeding, media changes, and other related needs. For suspension cells, devices may be provided within the incubator to separate cells from waste media (e.g., centrifuge(s) to facilitate cell pelleting) to facilitate automated media changes as part of an incubator provided herein. In some embodiments, the document provides a system comprising a cell culture incubator connected to a computer, capable of automatically monitoring and adjusting cell culture conditions for optimal growth of the cell culture.

In some embodiments, cells are passaged within an incubator described herein. In some embodiments, a cell culture is split and a subset of the cell culture is transferred to a fresh culture vessel for further growth. In some embodiments (e.g., for adherent cell cultures), cells are detached (e.g., mechanically, for example using gentle scraping, and/or enzymatically, for example using trypsin-EDTA or one or more other enzymes) from a surface prior to being transferred to a fresh culture vessel. In some embodiments (e.g., for suspension cell cultures), a small volume of a cell culture is transferred to a fresh culture vessel.

In some embodiments, cell cultures are manipulated in other ways during culture in incubators and vessels described herein. For example, cell cultures may be transfected with nucleic acids (e.g., DNA or RNA) or exposed to viral infection (e.g., using recombinant virus particles to deliver DNA or RNA).

Aseptic techniques can be used to prevent or minimize contamination of cell cultures during growth and manipulation. In some embodiments equipment (e.g., pipettes, fluid handling devices, manipulating devices, other automated or robotic devices, etc.) that is used for cell culture is sterilized using an appropriate technique. Non-limiting techniques include heat exposure (e.g., autoclaving), surface disinfection (e.g., using alcohol, bleach, or other disinfectant), irradiation, and/or exposure to a disinfectant gas (e.g., ozone, hydrogen peroxide, etc.) as described herein. In some embodiments, media is sterilized using an appropriate technique. Non-limiting techniques include heat exposure (e.g., autoclaving), antimicrobial/antiviral treatment, filtration, and/or irradiation.

In some embodiments, manipulations of cell cultures are performed under aseptic conditions, for example in an environment (e.g., within an incubator chamber) that has been disinfected and in which the air has been filtered to remove potential contaminants.

In some embodiments, cell cultures are grown and maintained under GMP-compliant conditions, including using GMP-compliant media or GMP-compliant liquid handling equipment and performing methods in conjunction with standard operation procedures (SOPs).

In some embodiments, cell cultures can be monitored and/or evaluated to detect contamination. In some embodiments, contamination by cells from a different type of organism can be detected. In some embodiments, contamination of a mammalian cell culture by mycoplasma, bacteria, yeast, or viruses can be detected using any suitable technique. In some embodiments, cell culture contamination can be detected by assaying for changes or for rates of change of one or more culture properties such as pH, turbidity, etc., that are characteristic of contamination (e.g., by bacteria or yeast) and not characteristic of the cells being grown in culture (e.g., mammalian cells). In some embodiments, one or more molecular detection assays (e.g., PCR, ELISA, RNA labeling, or other enzymatic techniques) or cell-based assays can be used to detect contamination (e.g., mycoplasma, bacterial, yeast, viral, or other contamination).

In some embodiments, cell cultures can be monitored and/or evaluated to detect contamination with cells of similar types (e.g., a human cell line contaminated by different human cells or by different mammalian cells). In some embodiments, cell cultures and their potential contamination can be evaluated using DNA sequencing or DNA fingerprinting (e.g., short tandem repeat-STR-fingerprinting), isoenzyme analysis, human lymphocyte antigen (HLA) typing, chromosomal analysis, karyotyping, cell morphology, or other techniques.

In some embodiments, cells produced using devices and methods provided herein can be frozen to preserve them for later use and/or for transport. In some embodiments, cells are mixed with a cryopreservation composition after growth and/or differentiation and prior to freezing. A cryopreservation composition can be added to a cell culture vessel or cells can be transferred from a cell culture vessel to a cryopreservation vessel along with a cryopreservation composition. Non-limiting examples of cryoprotectants that can be included in a cryopreservation composition include DMSO, glycerol, PEG, sucrose, trehalose, and dextrose. In some embodiments, a freezer may be present in or in proximity with an incubator to facilitate freezing of cells isolated from cell cultures.

### Cell Culture Incubators:

This document relates to incubators and methods for culturing, manipulating, and/or monitoring cells under controlled conditions (e.g., under aseptic and/or sterile conditions). In some embodiments, the cell culture incubators provided herein include an incubator cabinet defining an internal chamber for incubation of cells in one or more cell culture vessels, in which the internal chamber is configured to hold the one or more cell culture vessels. In some cases, in addition to an internal door from the transfer chamber to the internal chamber, the incubators include at least one external door (e.g., 1, 2, 3, 4, or more external doors) opening from an external environment directly to the internal chamber, for example to provide alternative access to the internal chamber during periods of time when the incubator is not operational, e.g., during maintenance of the incubator. In some embodiments, the incubators include a storage location within the internal chamber for storing one or more cell culture vessels. In some embodiments, a cell culture vessel transfer device is provided in the incubator for moving one or more cell culture vessels from a first imaging location to a storage location and/or from a storage location to an first imaging location.

In some embodiments, incubators or incubator cabinets provided herein are rectangularly cuboidal in shape. In some embodiments incubators or incubator cabinets provided herein have a rectangular footprint in a range of 1 ft² to 16 ft². In some embodiments incubators or incubator cabinets provided herein have a rectangular footprint of up to about 1 ft², 2 ft², 3 ft², 4 ft², 5 ft², 6 ft², 7 ft², 8 ft², 9 ft², 10 ft², 11 ft², 12 ft², 13 ft², 14 ft², 15 ft², or 16 ft². In some embodiments incubators or incubator cabinets provided herein have a total chamber volume in a range of 1 ft³ to 100 ft³. In some embodiments incubators or incubator cabinets provided herein have a chamber volume of up to about 1 ft³, 5 ft³, 10 ft³, 25 ft³, 50 ft³ or 100 ft³. In some embodiments incubators or incubator cabinets provided herein have a rectangular footprint in a range of 0.09 m² to 1.78 m². In some embodiments incubators or incubator cabinets provided herein have a rectangular footprint of up to about 0.1 m², 0.2 m², 0.3 m², 0.4 m², 0.5 m², 0.6 m², 0.7 m², 0.8 m², 0.9 m², 1.0 m², 1.1 m², 1.2 m², 1.3 m², 1.4 m², 1.5 m², 1.6 m², or 1.7 m². In some embodiments, incubators or incubator cabinets provided herein have a total chamber volume in a range of 0.03 m³ to 3 m³. In some embodiments incubators or incubator cabinets provided herein have a chamber volume of up to about 0.03 m³, 0.1 m³, 0.3 m³, 1 m³, or 3 m³.

### Materials

In some embodiments, an incubator cabinet is single-walled. In some embodiments, an incubator is double-walled. In some embodiments, insulation material is provided between the double walls of an incubator cabinet to control heat loss from the cabinet and facilitate temperature control in the cabinet. In some embodiments, the outer wall of an incubator cabinet includes a sheet metal, e.g., a 14-20 gauge cold rolled steel. In some embodiments, an inner wall (e.g., a chamber surface) of an incubator cabinet includes electro-polished stainless steel. In some embodiments, an inner wall (e.g., a chamber surface) of an incubator cabinet includes corrosion resistant materials, such as, titanium, cobalt-chrome, tantalum, platinum, zirconium, niobium, stainless steel, and alloys thereof. However, in some embodiments, a chamber surface of an incubator cabinet includes a polymeric material such as polytetrafluoroethylene (PTFE), or a polymeric material know under the trade name of Parylene. In some embodiments, a chamber surface may have anti-microbial properties, such as copper or silver or anti-microbial compounds incorporated into a polymeric surface coating.

### Monitoring Equipment

In some embodiments, the environment inside an incubator is controlled by a control system that may be configured to control the temperature, humidity, carbon dioxide, oxygen, and other gaseous components (e.g., sterilization gases, such as, ozone, and hydrogen peroxide) inside the incubator (e.g., in one or more internal chambers). In some embodiments, a control system controls the environmental conditions (e.g., temperature, humidity, carbon dioxide, oxygen and other gaseous components) within each internal chamber separately. For example, in order to protect sensitive mechanical, electronic and optical components, the humidity of an internal chamber may be maintained at a lower level than an internal chamber having a storage location. In some embodiments, the incubator is further provided with a monitoring system with predefined sensors. Examples of monitoring devices include, but are not limited, to oxygen monitors, carbon dioxide monitors, ozone gas detectors, hydrogen peroxide monitors and multi gas monitors. For example, in some embodiments, an incubator advantageously includes a plurality of sensors responsive to different parameters relevant to cell growth, which may include temperature, air purity, contaminant levels, pH, humidity, N₂, CO₂, O₂, and light. By means of this monitoring system, parameters in the incubator can be measured using sensors for the duration of a culture or process. In some embodiments, parameters measured by the sensors are transmitted by the monitoring system via a line to a computer-controlled monitoring and control system for further processing as discussed herein.

In some embodiments, an environmental monitoring system can be used in conjunction with an incubator described herein. In some embodiments, one or more sensors that provide for the measurement of temperature, air composition (e.g., CO₂ concentration, O₂ concentration, etc.), and/or humidity of the system can be associated with an incubator (e.g., fitted within an incubator cabinet). In some embodiments, one or more such sensors can be incorporated as part of an incubator (e.g., attached to, integral to, or otherwise connected to an internal wall or door of the incubator). In some cases, one or more sensors can be positioned at any suitable location(s) outside or inside an incubator cabinet (e.g., within a transfer chamber and/or an internal chamber, for example attached to an internal wall, and/or upper or lower internal surface).

In some embodiments, a gas sensor is provided that can provide a reading in real time of the concentration of gas in contact with the sensor (e.g., gas in a cabinet, or ambient air) in percent, parts per million, or any other standard unit. Gas sensors for use in the methods and incubators provided herein include CO₂ sensors, O₂ sensors, N₂ sensors, ozone gas detectors, hydrogen peroxide monitors, multi gas monitors, and CO sensors. Such sensors are available from a number of commercial sources. In some cases, the environment of the incubator may be modulated or controlled based upon the information provided by the sensors described herein. For example, the level of CO₂ in an incubator may be increased upon indication from a CO₂ sensor that a lower than desirable concentration of CO₂ is present in the incubator.

In some embodiments, one or more heating or cooling elements can be incorporated within the incubator (e.g., on an inner surface of the cabinet or door, and/or integrated within one or more of the walls and/or the base of the cabinet) for purposes of controlling the temperature within the incubator. In some embodiments, a heating element can be used for thawing liquids, for example, cell culture media or other reagents.

In some embodiments, one or more air or oxygen sources, carbon filters, and/or one or more humidification or dehumidification systems are connected to the incubator and configured to control the level of oxygen, carbon dioxide, and/or humidity within the incubator (e.g., in response to signals from the one or more sensors in or attached to the incubator). In some embodiments, one or more controllers are attached to the sensors and other systems to control the internal environment of the incubator.

In some embodiments, an incubator can include one or more light sources (e.g., an incandescent bulb, LED, UV or other light source). These can be placed within the incubator to illuminate regions within the cabinet. In some embodiments, the culture system operation is monitored using a camera or other light sensitive device that can be placed within or outside the incubator. In some embodiments, the light source is a sterilizing light source. For example, a UV lamp may be located within the transfer chamber and/or the interior chamber of the incubator.

In some embodiments, the incubator includes a transparent object (e.g., window) that allows visible light or other light wavelengths from within the incubator to be detected by a camera or other light sensitive device placed outside the incubator. In some embodiments, the inner surface of the transparent object can be wiped (e.g., from the inside of the cabinet) to prevent or remove condensation droplets that may accumulate (e.g., due to the humid air inside the incubator) on the inner surface and interfere with the monitoring of the system. In some embodiments, the surface can be wiped by a wiper that is automatically controlled by a controller.

### Seals

In some embodiments, a culture cabinet includes windows, doors, or openings that when closed are sealed to preserve sterility after the incubator cabinet has been sterilized. In some embodiments, each seal of the incubator cabinet is air tight up to a threshold level of pressure (e.g., up to 1 atm). In some embodiments, a gasket is provide to ensure a desired level of sealing capacity. In general, a “gasket” is understood as a mechanical seal that fills the space between two objects, generally to prevent leakage between the two objects while under compression. Gaskets are commonly produced by cutting from sheet materials, such as gasket paper, rubber, silicone, metal, cork, felt, neoprene, nitrile rubber, fiberglass, or a plastic polymer (such as polychlorotrifluoro-ethylene). It is often desirable that a gasket be made from a material that provides some degree of yielding such that it is able to deform and tightly fills the space it is designed for, including any slight irregularities. In some embodiments, gaskets can be used with an application of sealant directly to the gasket surface to function properly. In some embodiments, a gasket material can include a closed-cell neoprene foam which is non-reactant with carbon dioxide or ozone.

### Transfer Devices

Incubators disclosed herein typically include one or more transfer devices for moving one or more items, e.g., from a first location to a second location, within the incubators. In some embodiments, the one or more items are one or more cell culture vessels (e.g., vessel (500)). In other embodiments, the one or more items are useful for maintenance of one or more cell culture vessels and include, but are not limited to, pipettes, capillaries, liquids (e.g., cell culture medium), nutrients, and other materials. In some embodiments, a transfer device includes a robotic arm. In some embodiments, the robotic arm includes a platform within an incubator cabinet that may move along a rail or conveyor running in various directions along an inner surface (e.g., inner wall, base, etc.) of incubator cabinet. In some embodiments, an incubator cabinet may be configured with more than one (e.g., 2, 3, 4, or 5, or more) robotic arms to increase the throughput of the instrument and to provide redundancy in the event that one of the robotic arms fail.

In some embodiments, a transfer device further includes a gripper assembly coupled to a robotic arm. In some embodiments, the gripper assembly includes one or more grippers mounted on the end of the robotic arm, each gripper having two or more (e.g., 3, 4, 5, or more) gripper fingers. In some embodiments, each of the gripper fingers on the robotic arm has a groove, friction plate, rubber pad, or other gripping surface. The gripping surface can allow the fingers to grip and transport various types of containers (e.g., culture vessels) within the cabinets. In some embodiments, the robotic arm may have an absolute encoder either coupled to the gripper assembly, or the platform, or a separate absolute encoder for each of the gripper assembly and/or the platform to determine whether the robotic arm is in a position where it may be safely homed (e.g., returned to a resting or storage configuration and/or location or origin of an operational coordinate system) without hitting an obstruction.

In some embodiments, because it may be desirable in certain situations for the reach of the robotic arm not to extend to some areas of the incubator cabinet, the robotic arm may instead reach these locations by inserting a container into or removing a container from a shuttle or conveyor belt, located, for example, on the incubator cabinet floor or other surface that moves along an axis (e.g., x-axis, y-axis) and provides access to at least some of those locations to which the robotic arm cannot reach.

In some embodiments, an incubator cabinet is designed to be used in conjunction with an external assay or laboratory automation system. For example, in some embodiments, the incubator cabinet may have a door having an opening large enough to allow the gripper arm to pivot outside of the incubator cabinet with a sufficient reach for the fingers to transport culture vessels or other containers or components from a transport line of the laboratory automation system into the incubator cabinet or transport external assay components into and/or out of the incubator cabinet.

In some embodiments, a robotic arm is designed to carry, among other things, culture vessels, in which case movements of the robotic arm are controlled to prevent jerking or accelerations of such vessels or other movements which may cause the spilling of samples from the vessels. In some embodiments, a robotic arm is designed to carry, among other things, culture vessels, in which case movements of the robotic arm are controlled to prevent movement of such vessels in ways which cause newly plated cells to congregate/concentrate in specific areas of the culture vessel.

In some embodiments, because a robotic arm transports vessels or other containers between specific positions in the incubator cabinet, the robotic arm or other components of the incubator can be designed to track precisely where the vessels or other containers are is located. In some cases, in an incubator cabinet with which a robotic arm may be used, there are likely to be areas, such as where other components of the incubator cabinet or walls of the incubator cabinet are located, and thus where certain movements of the robotic arm may be limited. In these cases, a homing mechanism can be used for each of various motors of the arms (e.g., x-motor, theta-motor and z-motor) to position properly the robotic arm to a known location after it is powered up or if a robotic arm collides with another object before resuming operation.

In some embodiments, an uninterruptible power supply (“UPS”) is attached to or within the incubator cabinet, or contained with it, to allow for an orderly shut-down of incubator operations, including saving of various automation and sample information and the completion of any transport or transfer process that is underway (e.g., the transport of a container or vessel that is being carried by the robotic arm to its destination). The operator may be alerted to unauthorized opening of the incubator by an audible signal, a visual signal, an electronic signal (e.g., an email or a text message), or in some other manner.

In some embodiments, a sensor or other feature is provided to detect when one or more doors of an incubator are opened (e.g., when an incubator cabinet door, such as an external or internal door, is opened). Such features are useful because they allow operators to keep track of or be warned of any unscheduled or unauthorized openings of the incubator (e.g., the incubator cabinet) that could jeopardize sterility, spoil a production, compromise an assay or experiment, etc.

In some embodiments, a radiofrequency beacon or other signal source is located within the incubator (e.g., within the incubator cabinet) that can be used to determine the location of one or more devices within the incubator cabinet (e.g., devices having sensors that can detect the signal and use it to determine their location). In some embodiments, the devices could have signal sources and the sensor(s) could be located within one or more of the chambers of an incubator cabinet (e.g., located on an internal surface of an internal chamber).

In some embodiments, light signals or lasers (e.g., a grid of laser signals) can be used to determine the location of one or more devices or components within the incubator cabinet. Such information can be communicated, e.g., wired or wirelessly, to an external computer or monitoring station. The information can be used to control operation of a transfer device, e.g., a robotic arm, within the incubator cabinet to ensure that the transfer device can grab, manipulate, or maneuver devices or items appropriately within the incubator cabinet.

In some embodiments, before containers or vessels are brought into an incubator cabinet, a user can select an automation system protocol based on the particular containers, vessels, ingredients, or cells that are being inserted into the incubator cabinet. Relevant information related to the incubator and/or one or more incubator components, and the cells being grown can be entered into a data system. For example, one or more identifiers such as barcodes (e.g., 1D or 2D barcodes) can be placed on the container or vessel, and other significant information, such as, the type of container, the contents of the container, what assays or manipulations are to be performed on the sample in the container can be specified. In some embodiments, information related to the incubator system and/or cells can be contained in one or more barcodes, on a separate data system, or a combination thereof. The user may also enter information that identifies the dimensionality (e.g., height, diameter) of the vessel or other container or the system itself can be configured to determine the height or other dimensions of the vessel or other container. Using this information, the robotic arm may be requested to transport a particular container, such as when an analytical module is ready to perform an assay or other manipulation on cells grown in the vessels or has completed performing an assay or manipulation.

### Computer and Control Equipment

The incubators provided herein include several components, including sensors, environmental control systems, robotics, etc. which may operate together at the direction of a computer, processor, microcontroller or other controller. The components may include, for example, a transfer device (e.g., robotic arm), a liquid handling devices, a delivery system for delivering culture vessels, a manipulator attached to the disclosed pipette tip, or other components to or from the incubator cabinet, an environmental control system for controlling the temperature and other environmental aspects of the incubator cabinet, a door operation system, an imaging or detection system, and a cell culture assay system.

In some cases, operations such as controlling operations of a cell culture incubator and/or components provided therein or interfacing therewith may be implemented using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format.

A computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable, mobile or fixed electronic device, including the incubator itself.

In some cases, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. In other examples, a computer may receive input information through speech recognition or in other audible format, through visible gestures, through haptic input (e.g., including vibrations, tactile and/or other forces), or any combination thereof.

One or more computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools, and may be compiled as executable machine language code or intermediate code that is executed on a framework or virtual machine.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein. In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

In some embodiments, information related to the operation of the incubator (e.g., temperature, humidity, gas composition, images, cell culture conditions, etc., or any combination thereof) can be obtained from one or more sensors associated with the incubator (e.g., located within the incubator cabinet, or located within the incubator but outside the incubator cabinet), and can be stored in computer-readable media to provide information about conditions during a cell culture incubation. In some embodiments, the readable media comprises a database. In some embodiments, said database contains data from a single incubator. In some embodiments, said database contains data from a plurality of incubators. In some embodiments, data is stored in a manner that makes it tamper-proof. In some embodiments, all data generated by the instrument (e.g., an incubator) is stored. In some embodiments, a subset of data is stored.

In some embodiments, the component (e.g., a computer) controls various processes performed inside the incubator. For example, a computer may direct control equipment (e.g., a manipulator, an imager, a fluid handling system, etc.). In some embodiments, the computer controls imaging of cell cultures, picking of cells, weeding of cells (e.g., removal of cell clumps), monitoring of cell culture conditions, adjustment of cell culture conditions, tracking of cell culture vessel movement within the incubator, and/or scheduling of any of the foregoing processes.

### Cell Assays

In some embodiments, incubator cabinets provided herein are configured with a microscope or other imager or other device for purposes of monitoring cell growth, viability or other aspect of cells. In some embodiments, the microscope or imager is used in conjunction with an assay performed within the incubator cabinet, such as an image based phenotypic screen or assay.

In certain embodiments, incubators provided herein are configured to permit one or more assays to be performed within an incubator cabinet or within a chamber operably connected to an incubator cabinet, e.g., a separate assay chamber that is part of the incubator. In some embodiments, incubators provided herein are configured to permit performance of a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, a nuclear fragmentation assay, or a combination thereof. Other exemplary assays include BrdU, EdU, or H3-thymidine incorporation assays; DNA content assays using a nucleic acid dye, such as Hoechst Dye, DAPI, actinomycin D, 7-aminoactinomycin D, or propidium iodide; cellular metabolism assays such as AlamarBlue, MTT, XTT, and CellTitre Glo; nuclear fragmentation assays; cytoplasmic histone associated DNA fragmentation assays; PARP cleavage assays; and, TUNEL staining assays.

### Treatments and Experimental Interventions

In certain embodiments, incubators provided herein are configured to permit high-throughput screening (HTS) within an incubator cabinet. In some embodiments, HTS refers to testing of up to, for example, 100,000 compounds per day. In some embodiments, screening assays may be carried out in a multi-well format, for example, a 96-well, 384-well format, or 1,536-well format, and can be performed using automated protocols. In such high throughput assays, it is possible to screen several thousand different compounds or compositions in a single day. In particular, each well of a microtiter plate can be used to run a separate assay against a selected test compound, or, if concentration or incubation time effects are to be observed, a plurality of wells can contain test samples of a single compound. It is possible to assay many plates per day; assay screens for up to about 6,000, 20,000, 50,000, or more than 100,000 different compounds are possible using the assays. Typically, HTS implementations of the assays described herein involve the use of automation. In some embodiments, an integrated robot system that includes of one or more robotic arms transports assay microplates between multiple assay stations for compound, cell, and/or reagent addition, mixing, incubation, and finally readout or detection. In some aspects, an HTS assay may include preparing, incubating, and analyzing many plates simultaneously, further speeding the data-collection process.

In some embodiments, assays can include test cells, control cells, and one or more test compounds, e.g., 10, 100, 1000, 10,000, or more test compounds. The cells and test agents can be arranged in one or more vessels in a manner suitable for assessing effect of the test compound(s) on the cells. These assays can be performed within one or more incubator cabinets of one or more incubators described herein. Typically, the vessels contain a suitable tissue culture medium, and the test compounds are present in the tissue culture medium and may be delivered to the culture medium within an incubator cabinet of an incubator provided herein in an automated fashion. A medium appropriate for culturing a particular cell type can be selected for use. In some embodiments, a medium is free or essentially free of serum or tissue extracts, while in other embodiments such a component is present. In some embodiments, cells are cultured on a plastic or glass surface.

The above aspects and embodiments may be employed in any suitable combination, as the present invention is not limited in this respect.

It should be understood that aspects of the invention are described herein with reference to certain illustrative embodiments and the figures. The illustrative embodiments described herein are not necessarily intended to show all aspects of the invention, but rather are used to describe a few illustrative embodiments. Thus, aspects of the invention are not intended to be construed narrowly in view of the illustrative embodiments. In addition, it should be understood that aspects of the invention may be used alone or in any suitable combination with other aspects of the invention.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A vessel for culturing cells, the vessel comprising:
a vessel body arranged to hold a volume of liquid containing cells to be cultured, the vessel body having a deformable portion arranged to produce movement of the volume of liquid sufficient to maintain the cells in suspension.

2. The vessel of claim 1, wherein the vessel body comprises a base and a sidewall.

3. The vessel of claim 1, wherein the deformable portion is located on at least one of the base and sidewall.

4. The vessel of claim 1, wherein the deformable portion is integrally formed with the vessel body.

5. The vessel of claim 1, wherein the deformable portion is removably attachable to the vessel body.

6. The vessel of claim 3, wherein a shape of the deformable portion is one of circular, ovular, rectangular, triangular and square.

7. The vessel of claim 1, wherein, in response to a deformation of the deformable portion, the volume of liquid is moved.

8. The vessel of claim 1, wherein the deformable portion is deformed in a direction towards an interior of the vessel body.

9. The vessel of claim 1, further comprising a second deformable portion.

10. The vessel of claim 9, wherein the deformable portion is located on one of the base and sidewall of the vessel body and the second deformable portion is located on the other of the base and sidewall of the vessel body.

11. The vessel of claim 1, wherein the deformable portion is arranged to produce movement of the volume of liquid in response to a pattern of deformation.

12. The vessel of claim 1, in combination with a deformation plate having one or more moveable members configured to deform the deformable portion to produce the movement of the volume of liquid.

13. A vessel for culturing cells, the vessel comprising:
a vessel body configured to hold a volume of liquid containing cells to be cultured, the vessel body having a base and a sidewall, at least a portion of the base being deformable;
wherein, in response to a pattern of deformation in the base, the cells are maintained in suspension while minimizing a shear stress applied to the cells.

14. The vessel of claim 13, wherein an entire portion of the base is deformable.

15. The vessel of claim 13, wherein the pattern of deformation includes one of a wave, circular, and serpentine pattern.

16. The vessel of claim 13, wherein, in response to a pattern of deformation at the base, the deformable portion is deformed in a direction towards the interior of the vessel body.

17. The vessel of claim 13, wherein at least a portion of the sidewall is deformable.

18. The vessel of claim 13, wherein the vessel body is cylindrically shaped.

19. A system for culturing cells, the system comprising:
a vessel having a body configured to hold a volume of liquid containing cells to be cultured, the vessel body having a deformable portion; and
one or more moveable members arranged to deform the deformable portion to produce a movement of the volume of liquid sufficient to maintain the cells in suspension.

20. The system of claim 19, further comprising a deformation plate having a housing and a top surface, wherein the moveable members are disposed in the housing.

21. The system of claim 20, wherein the one or more moveable members move to a forward position to contact and deform the deformable portion.

22. The system of claim 21, wherein, in the forward position, the one or more moveable members extend beyond a plane extending through the top surface.

23. The system of claim 19, further comprising a controller, wherein the controller instructs the one or more moveable members to deform the deformable portion.

24. The system of claim 19, further comprising an actuator arranged to move the one or more moveable members.

25. The system of claim 23, wherein the controller instructs the one or more moveable members to perform a sequence of deformations.

26. The system of claim 23, wherein the one or more moveable members are arranged to create a deformation pattern in the deformable portion.

27. The system of claim 26, wherein the deformation pattern includes one of a wave, circular, and serpentine pattern.

28. A method of culturing cells, the method comprising:
placing a volume of liquid containing cells to be cultured in a vessel having a deformable portion; and
deforming the deformable portion to produce a movement of the volume of liquid sufficient to maintain the cells in suspension.

29. The method of claim 28, wherein deforming the deformable portion comprises manually deforming the deformable portion.

30. The method of claim 28, further comprising placing the vessel on a deformation plate having one or more moveable members; wherein deforming the deformable portion comprises deforming the deformable portion with the one or more moveable members.

31. The method of claim 28, wherein placing a volume of liquid containing cells to be cultured in a vessel having a deformable portion comprises placing a volume of liquid containing cells to be cultured in a vessel having a deformable portion on at least one of a sidewall and base of a vessel body.

32. The method of claim 30, wherein deforming the deformable portion with the one or more moveable members comprises moving the one or more moveable members to a forward position that extends beyond a plane extending through the top surface.

33. The method of claim 30, wherein deforming the deformable portion with the one or more moveable members comprises contact the deformable portion with the one or more moveable members to move the deformable member towards an interior of a vessel body.

34. The method of claim 30, wherein deforming the deformable portion with the one or more moveable members comprises creating a deformation pattern in the deformable portion.
